(19) United States
(12) Patent Application Publication
(10) Pub. No.: US 2017/0087007 A1
(43) Pub. Date: Mar. 30, 2017

(54) COOLING AND WARMING PROTECTOR

(71) Applicant: Cheng-Chuan Yang, Taichung City (TW)

(72) Inventor: Cheng-Chuan Yang, Taichung City (TW)

(21) Appl. No.: 14/870,010

(22) Filed: Sep. 30, 2015

(51) Int. Cl. A61F 7/02; A61H 23/02; A61F 7/00; A41D 13/05; A41D 13/005

(52) U.S. Cl. CPC A61F 7/02; A41D 13/05; A41D 13/0051; A41D 13/0053; A61F 7/007; A61H 23/02; A61F 2007/0056; A61F 2007/0086; A61F 2007/0096; A61F 2007/0231; A61F 2007/0295; A61F 2007/0032; A61F 2007/0042; A61F 2007/0027; A61F 2007/003; A61H 2201/0207; A61H 2201/0214

(57) ABSTRACT

A protector includes a body having a cooling/warming device and a connection device. The cooling/warming device includes a bag, a circulation device and a chip, wherein the circulation device is connected to the bag and the chip. Liquid is received in the water bag and circulates within the circulation device, and the chip changes the temperature of the liquid in the circulation device. The connection device is easily attached to the user's body and the temperature of the liquid is set to provide cooling or warming features to the muscles of the user's body.

### BACKGROUND OF THE INVENTION

1. Fields of the Invention

The present invention relates to a protector, and more particularly, to a protector for providing cooling or warming features.

2. Descriptions of Related Art

The conventional protectors for protecting elbows, knees, wrists, shoulders, neck or other parts of athletes generally provide a pad which includes parts that provide warming or cooling feature so as to reduce injury to the muscles of the specific portions of the athlete' body. The protectors provide cooling feature when the muscles are injured at the first stage, and provide warming feature after the injured muscles are applied the first-stage treatment. Generally, most of the protectors are designed to provide warming feature and includes multiple heat generating member and multiple conductive members. The power source is electrically connected to the conductive members which are connected to the heat generating members. The electric power activate the heat generating members to provide warming feature. However, most of the existed protectors do not have both warming and cooling features, therefore, a cooling protector has to be purchased separately.

The present invention intends to provide a cooling and warming protector to eliminate the shortcomings mentioned above.

### SUMMARY OF THE INVENTION

The present invention relates to a protector and comprises a body having a cooling/warming device and a connection device. The cooling/warming device has a bag, a circulation device and a chip, wherein the circulation device is connected to the bag and the chip. Liquid is received in the water bag and circulates within the circulation device. The chip changes temperature of the liquid in the circulation device.

Preferably, the cooling/warming device has a motor for pumping the liquid, a temperature controller, a temperature detector, a circuit unit and a power source. The motor is connected to the circulation device and the chip. The temperature controller and the temperature detector are connected to the circuit unit which is connected to the power source. A heat dissipating member is connected to one side of the chip. A switch and a temperature regulating member are connected to the outside of the body. The switch is electrically connected to the circuit unit. The temperature regulating member is electrically connected to the temperature controller. When the switch is turned on, the motor is activated via the circuit unit and pumps the liquid in the bag to circulate the liquid within the circulation device. The temperature of the liquid in the circulation device is changed by the chip and the liquid returns to the bag via the circulation device. The temperature detector detects the temperature of the liquid in the circulation device. The temperature regulating member is operated to control the temperature controller to set the temperature of the liquid in the circulation device by the circuit unit and the chip. The heat dissipating member absorbs heat from the chip.

Preferably, the body has multiple vibration motors which are connected to the circulation device and electrically connected to the circuit unit. Each vibration motor is connected with a soft pad.

Preferably, the connection device includes a hook surface and a loop surface which is connected to the hook surface.

Preferably, the body has an opening.

Preferably, a zipper is connected to the opening, and two zipper sliders are respectively connected to two ends of the zipper so as to control the size of the opening.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 shows the parts of the protector of the present invention;

FIG. 2 is a side view of the protector of the present invention;

FIG. 3 is a perspective view to show the protector of the present invention;

FIG. 4 shows that the protector of the present invention is use to the user's elbow;

FIG. 5 shows that the protector of the present invention is use to the user's knee;

FIG. 6 shows that the protector of the present invention is use to the user's waist;

FIG. 7 shows the second embodiment of the protector of the present invention;

FIG. 8 shows that the second embodiment of the protector of the present invention is used to the user's shoulder;

FIG. 9 shows that the second embodiment of the protector of the present invention is used to the user's knee;

FIG. 10 shows the third embodiment of the protector of the present invention;

FIG. 11 shows that the third embodiment of the protector of the present invention is used to the user's shoulder, and

FIG. 12 shows that the third embodiment of the protector of the present invention is used to the user's elbow.

### DETAILED DESCRIPTION OF THE PREFERRED EMBODIMENT

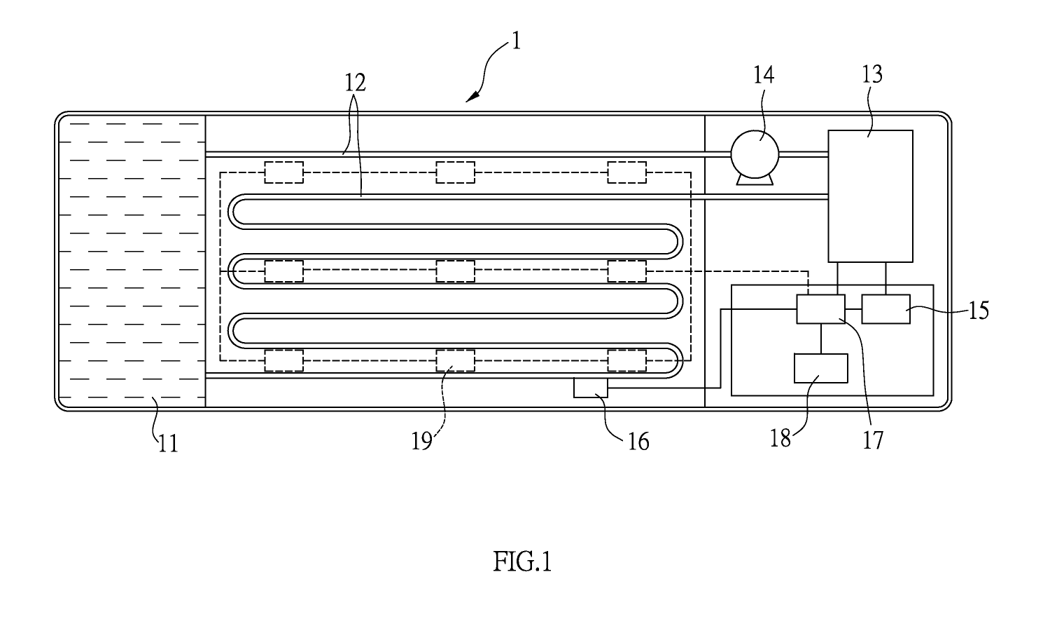

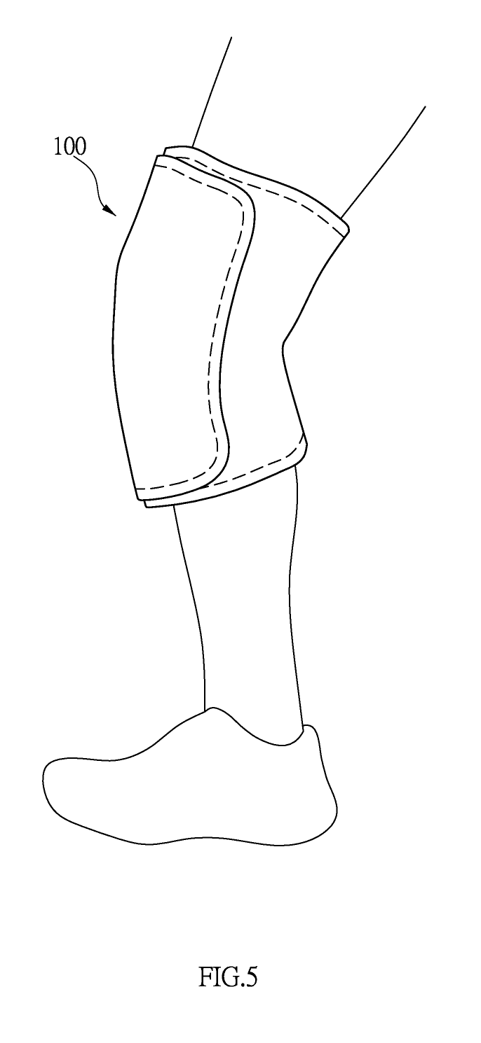

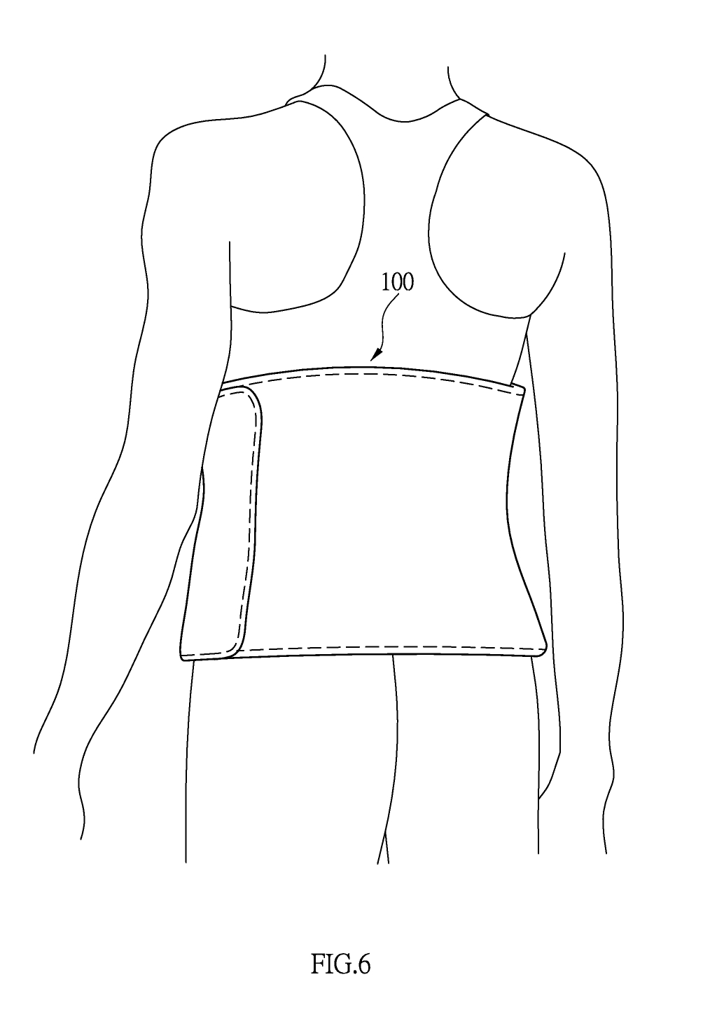

Referring to FIGS. 1 to 6, the protector of the present invention comprises a body 100 having a cooling/warming device 1 and a connection device 2. The cooling/warming device 1 has a bag 11, a circulation device 12 and a chip 13. The circulation device 12 is connected to the bag 11 and the chip 13. Liquid is received in the water bag 11 and circulates within the circulation device 12. The chip 13 changes the temperature of the liquid in the circulation device 12. The connection device 213 is used to attach the protector to the desired portion of the user's body.

As shown in FIGS. 1 to 3, the cooling/warming device 1 comprises a motor 14 for pumping the liquid, a temperature controller 15, a temperature detector 16, a circuit unit 17 and a power source 18. The motor 14 is connected to the circulation device 12 and the chip 13. The temperature controller 15 and the temperature detector 16 are connected to the circuit unit 17 which is connected to the power source 18. A heat dissipating member 3 is connected to one side of the chip 13. The body 100 further has a switch 4 and a temperature regulating member 5 connected to the outside thereof The switch 4 is electrically connected to the circuit unit 17. The temperature regulating member 5 is electrically connected to the temperature controller 15. When the switch 4 is turned on, the motor 14 is activated via the circuit unit 17 and pumps the liquid in the bag 11 to circulate the liquid within the circulation device 12. The temperature of the liquid in the circulation device 12 is changed by the chip 13 and the liquid returns to the bag 11 via the circulation device 12. The temperature detector 16 detects the temperature of the liquid in the circulation device 12, and the temperature regulating member 5 is operated to control the temperature controller 15 to set the temperature of the liquid in the circulation device 12 by the circuit unit 17 and the chip 13. The heat dissipating member 3 absorbs heat from the chip 13.

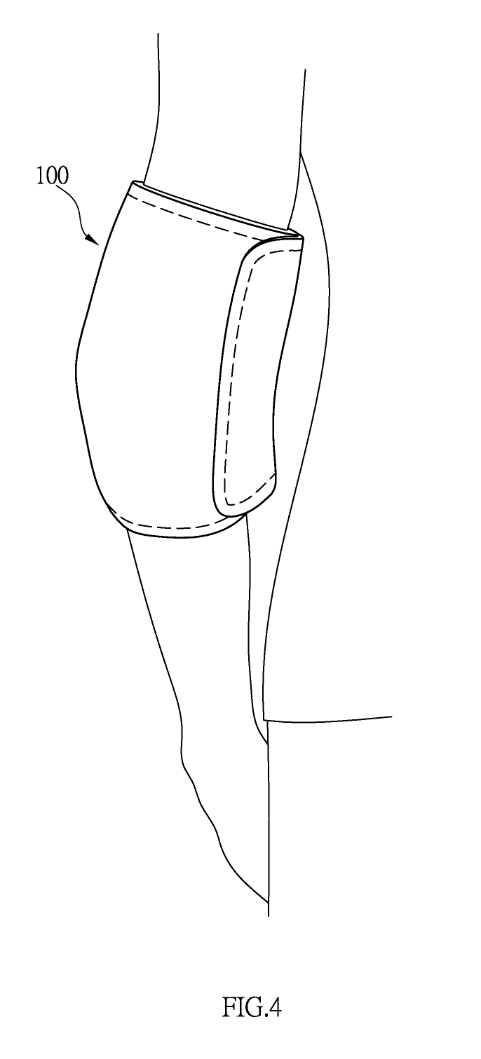

The protector of the present invention can be easily applied to the desired portions of the user's body such as the shoulders, the neck, the elbows, the waist, the arms, the back, the chest, the knees, the ankles and the feet as shown in FIGS. 4 to 6. The liquid circulates within the circulation device 12 by pumping the liquid from the bag 11 by the motor 14, and the chip 13 changes and sets the temperature of the liquid according the needs of the user by the temperature controller 15 and the temperature detector 16. When the desired temperature of the liquid is reached by the detection of the temperature detector 16, the circuit unit 17 controls the temperature controller 15 to control the chip 13 to maintain the temperature. Therefore, the protector can provide the user desired features.

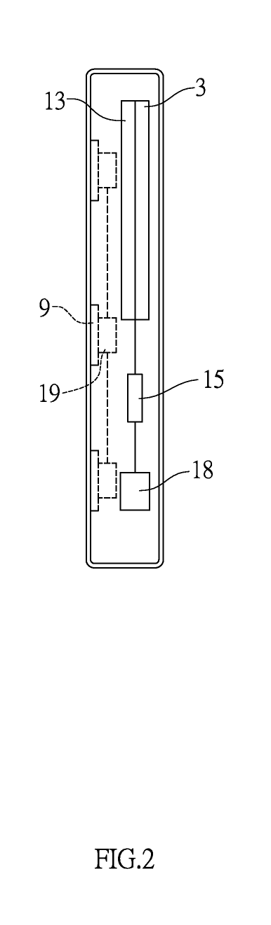

As shown in FIGS. 1 and 2, the body 100 further has multiple vibration motors 19 which are connected to the circulation device 12 and electrically connected to the circuit unit 17. The vibration motors 19 provide massaging feature to the user to enhance the blood circulation of the portion that needs to be treated. Each vibration motor 19 is connected with a soft pad 9 which provides a cushion feature when the vibration motors 19 are in operation. The pads 9 also make the wearing of the present invention be comfortable.

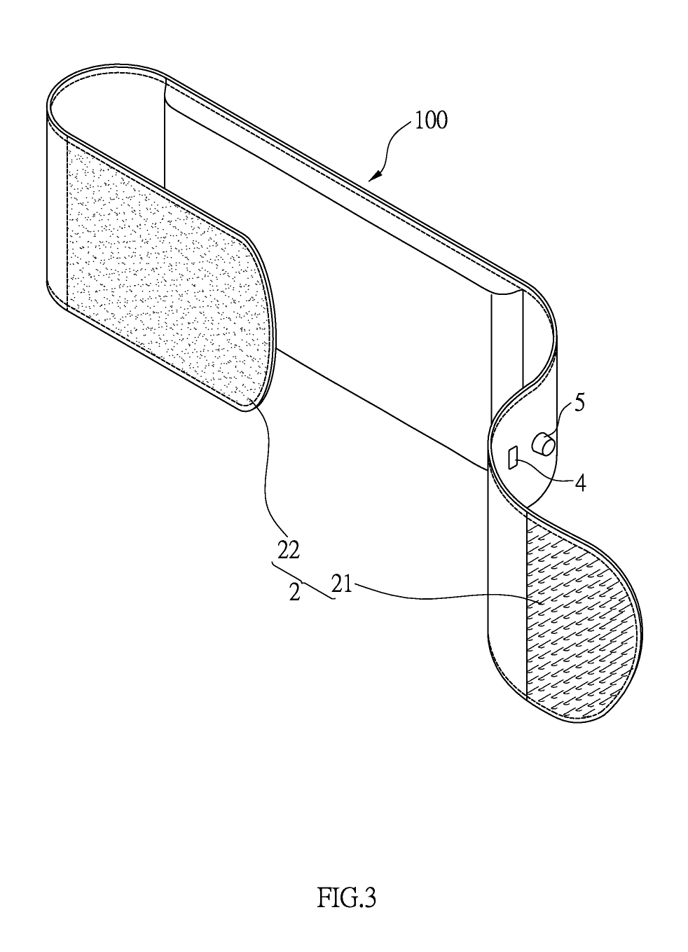

As shown in FIG. 3, the connection device 2 includes a hook surface 21 and a loop surface 22 which is connected to the hook surface 21 so as to securely position the protector of the present invention to the portion of the user's body.

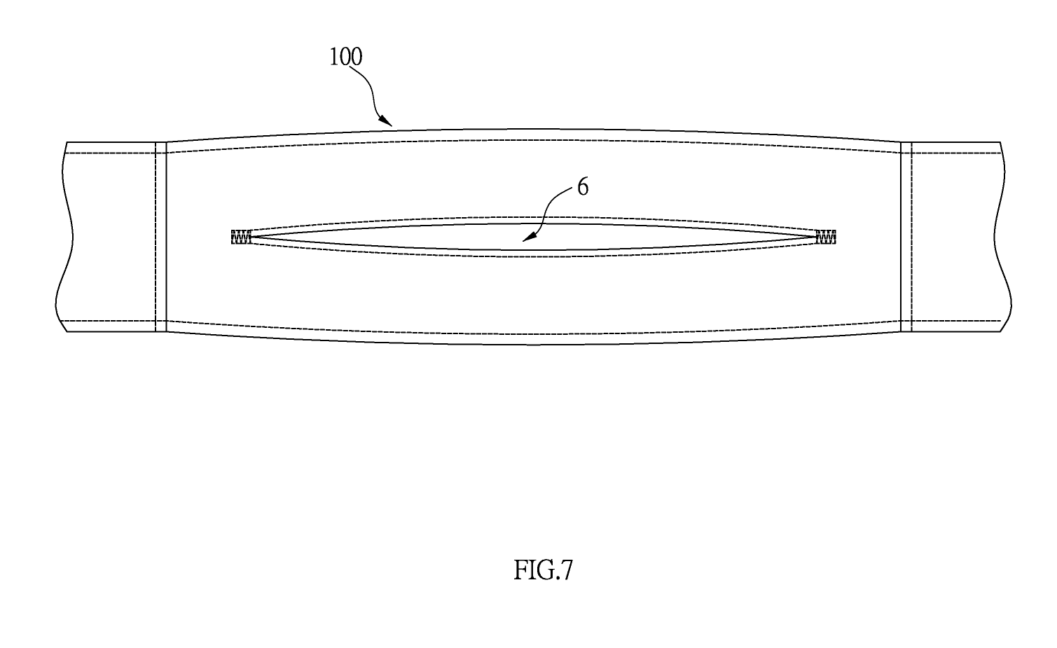

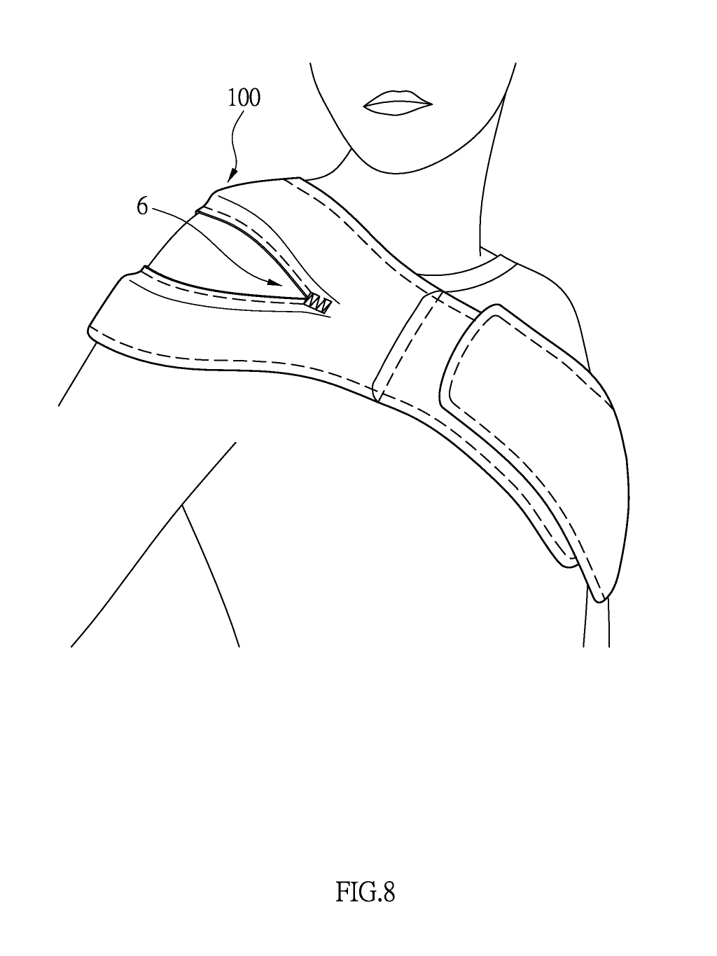

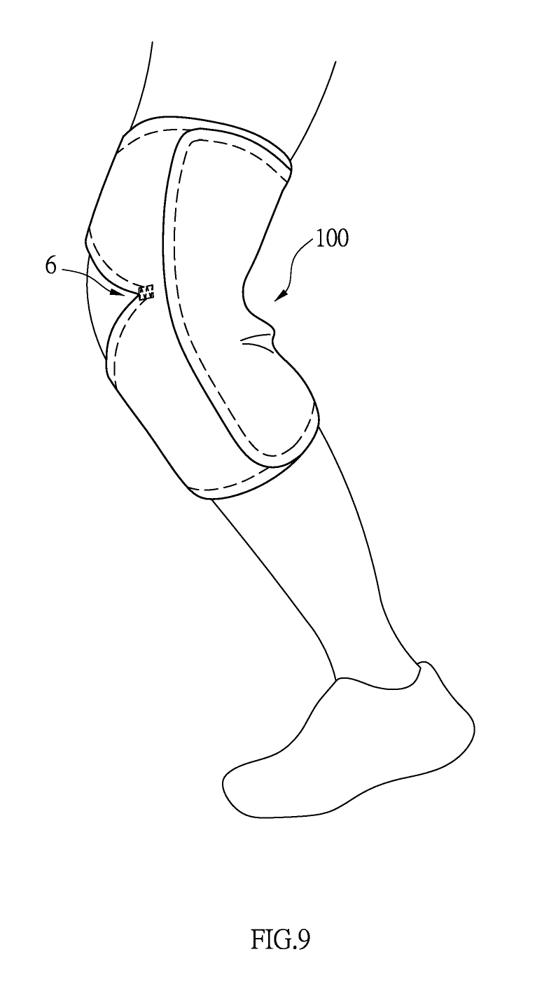

As shown in FIG. 7, the body 100 has an opening 6 which allows the elbow, shoulder or knee to freely move via the opening 6 as shown in FIGS. 8 and 9.

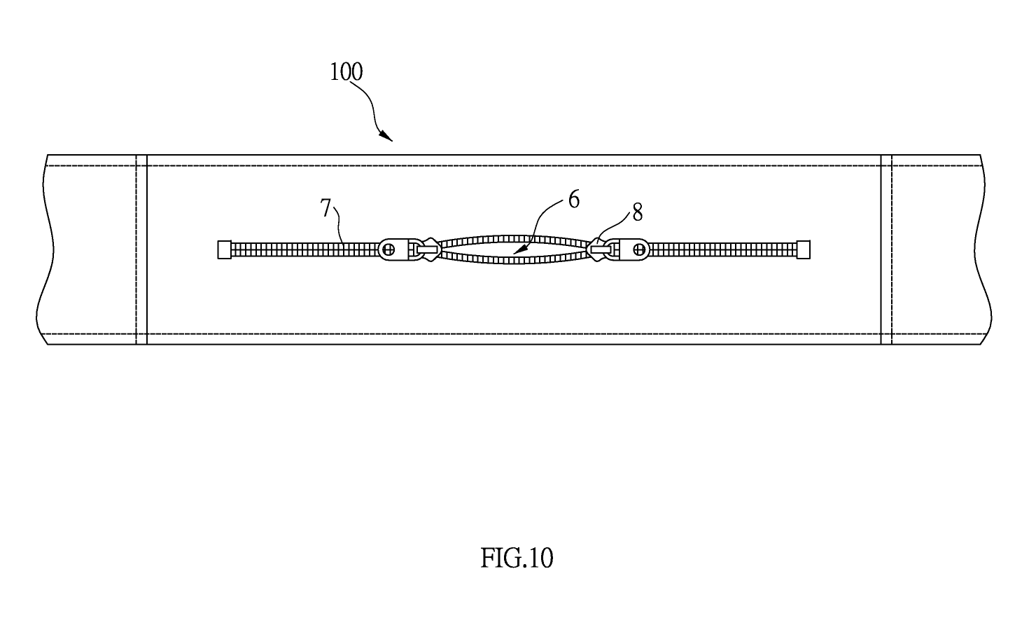

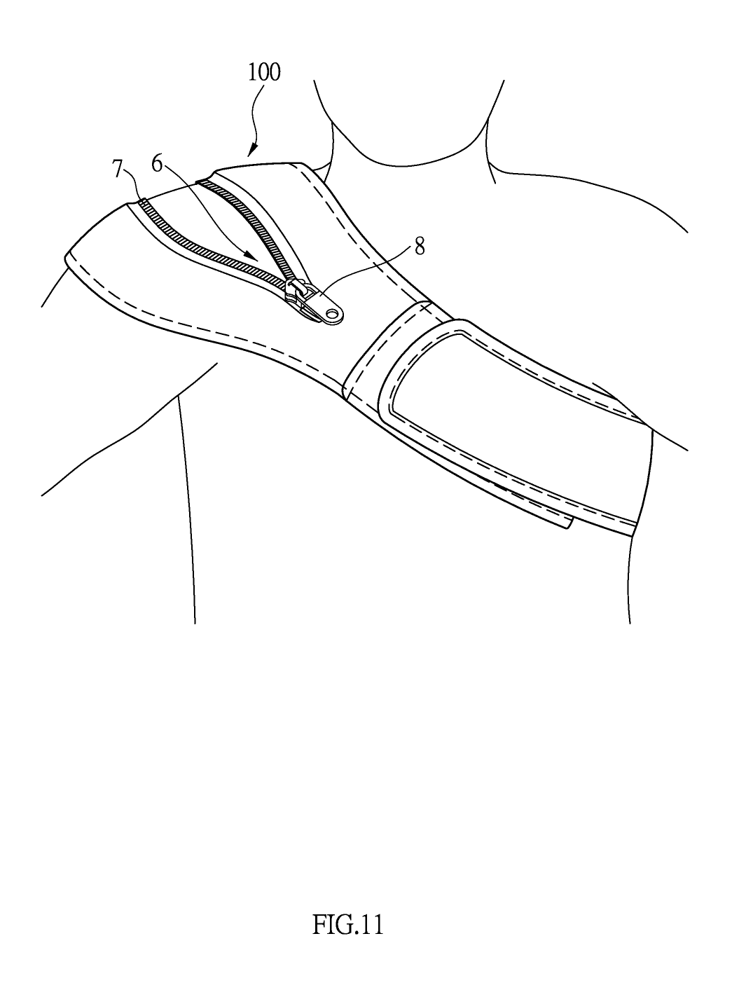

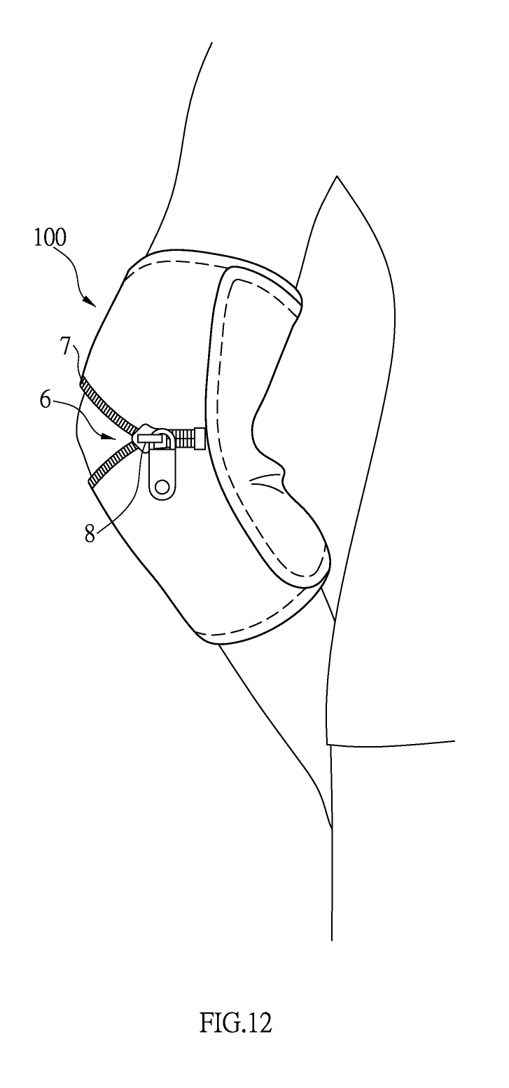

As shown in FIG. 10, a zipper 7 is connected to the opening 6, and two zipper sliders 8 are respectively connected to two ends of the zipper 7. By moving the two zipper sliders 8 toward each other or away from each other to control the size of the opening 6 to meet different requirements of practical needs as shown in FIGS. 11 and 12.

While we have shown and described the embodiment in accordance with the present invention, it should be clear to those skilled in the art that further embodiments may be made without departing from the scope of the present invention.

What is claimed is:

1. A protector comprising:
a body having a cooling/warming device and a connection device, the cooling/warming device having a bag, a circulation device and a chip, the circulation device connected to the bag and the chip, liquid received in the water bag and circulating within the circulation device, the chip changing temperature of the liquid in the circulation device.

2. The protector as claimed in claim 1, wherein the cooling/warming device has a motor for pumping the liquid, a temperature controller, a temperature detector, a circuit unit and a power source, the motor is connected to the circulation device and the chip, the temperature controller and the temperature detector are connected to the circuit unit which is connected to the power source, a heat dissipating member is connected to one side of the chip, the body has a switch and a temperature regulating member connected to an outside thereof, the switch is electrically connected to the circuit unit, the temperature regulating member is electrically connected to the temperature controller, when the switch is turned on, the motor is activated via the circuit unit and pumps the liquid in the bag to circulate the liquid within the circulation device, the temperature of the liquid in the circulation device is changed by the chip and the liquid returns to the bag via the circulation device, the temperature detector detects the temperature of the liquid in the circulation device, the temperature regulating member is operated to control the temperature controller to set the temperature of the liquid in the circulation device by the circuit unit and the chip, the heat dissipating member absorbs heat from the chip.

3. The protector as claimed in claim 2, wherein the body has multiple vibration motors which are connected to the circulation device and electrically connected to the circuit unit, each vibration motor is connected with a soft pad.

4. The protector as claimed in claim 1, wherein the connection device includes a hook surface and a loop surface which is connected to the hook surface.

5. The protector as claimed in claim 1, wherein the body has an opening.

6. The protector as claimed in claim 5, wherein a zipper is connected to the opening, two zipper sliders are respectively connected to two ends of the zipper.
